(19) United States
(12) Patent Application Publication
(10) Pub. No.: US 2006/0073493 A1
(43) Pub. Date: Apr. 6, 2006

(54) Method of sequencing a genome

(75) Inventors: Daniel P. Fasulo, Titusville, NJ (US); Robert A. Holt, North Vancouver (CA); Cheryl A. Evans, Cedar Hill, CA (US); Jeannine D. Gocayne, Potomac, MD (US); Peter W. Li, Brookeville, MD (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/153,991

(22) Filed: Jun. 15, 2005

Related U.S. Application Data
(60) Provisional application No. 60/579,742, filed on Jun. 15, 2004.

(51) Int. Cl. C12Q 1/68; G06F 19/00

(52) U.S. Cl. 435/6; 702/20

(57) ABSTRACT

A method and computer-program product for sequencing nucleic acid sequences using restriction fragment maps derived from end-sequenced nucleotide fragments. The initial nucleotide sequence can be processed to form a shot-gun-data set. The present teachings employ a technique called Restriction Site Shotgun Sequencing (RSSS.) It can reduce the amount of overlap required between fragment ends while still producing a good assembly. A decrease in overlap can be achieved by using additional information in the fragments to assist in determining that two fragments overlap.

### CROSS-REFERENCE TO RELATED APPLICATIONS

This application claims a priority benefit under 35 U.S.C. § 119(e) from U.S. Patent Application No. 60/579,742, filed Jun. 15, 2004, which is incorporated herein by reference.

### FIELD

The present teachings relate to the field of sequencing genetic material.

### BACKGROUND

Traditional shotgun sequencing forms scaffolds by examining the overlap between the sequenced ends of fragments. First, genetic sequence material is sheared into fragments. These fragments are size selected to isolate fragments of specific length; typically, 2 kbp, 10 kbp, and 150 kbp. Selected fragments are inserted into cloning vectors and cloned. After removal from clones, the first several hundred bases of each end of the insert sequence are determined. Next, algorithms determine fragment orientation and their relationship to each other utilizing fragment overlap and length information. Overlapping fragments are collapsed into a scaffold.

Generally, a significant number of bases between fragments must agree before it can be stated with a degree of certainty that fragments do in fact overlap. Generally, the number of fragments, and hence clones, required for sequencing is directly proportional to the amount of overlap required. For example, statistical calculations show that 5× sequencing coverage (50× clone coverage) is required for a “good” assembly (˜90% of all bases established.) Clone coverage is defined as the average number of clones that cover any particular base and sequencing coverage is defined as the average number of independently sequenced bases that are used to determine the consensus base. Thus for 5× coverage, on average 5 independently sequenced bases cover any base on the consensus sequence.

The present teachings employ a technique called Restriction Site Shotgun Sequencing (RSSS.) It can reduce the amount of overlap required between fragment ends while still producing a good assembly. A decrease in overlap can be achieved by using additional information in the fragments to assist in determining that two fragments overlap.

### SUMMARY

To be added after claims are finalized.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates the steps in traditional shotgun sequencing.

FIG. 2 illustrates the process contemplated by an embodiment of the present teachings.

FIG. 3 is a flowchart showing steps contemplated by an embodiment of the present teachings.

FIG. 4a illustrates a clone comprising, the vector, insert and restriction sites.

FIG. 4b illustrates the digestion products of the clone in FIG. 4a.

FIG. 5 illustrates a single base extension reaction on a sticky-end fragment.

FIG. 6 illustrates electrophoretic traces that can result from the separation of fragments.

FIG. 7 illustrates an embodiment of restriction fragment mapping being used in scaffold generation.

FIG. 8 illustrates expected fragment lengths for the CATG digest of cloning vector pBR196c.

FIG. 9 illustrates the frequency of fragment sizes of insert digest fragments.

FIG. 10a illustrates common fragment sizes for three clones starting at nonconcurrent positions.

FIG. 10b illustrates common fragment sizes for three clones starting at concurrent positions.

FIG. 11 illustrates an embodiment where graphing is used to determine an threshold total-length-of-common-fragments value.

FIG. 12 illustrates an embodiment that uses fragment length to determine fragment orientation.

FIG. 13 illustrates an embodiment that uses out of range fragment sizes to orient fragments.

FIG. 14 illustrates an embodiment that considers polymorphic sites when placing fragments.

FIG. 15 illustrates an embodiment that considers clones containing same sized fragments during tiling.

FIG. 16 is a block diagram that illustrates a computer system, according to various embodiments, upon which embodiments of the present teachings may be implemented.

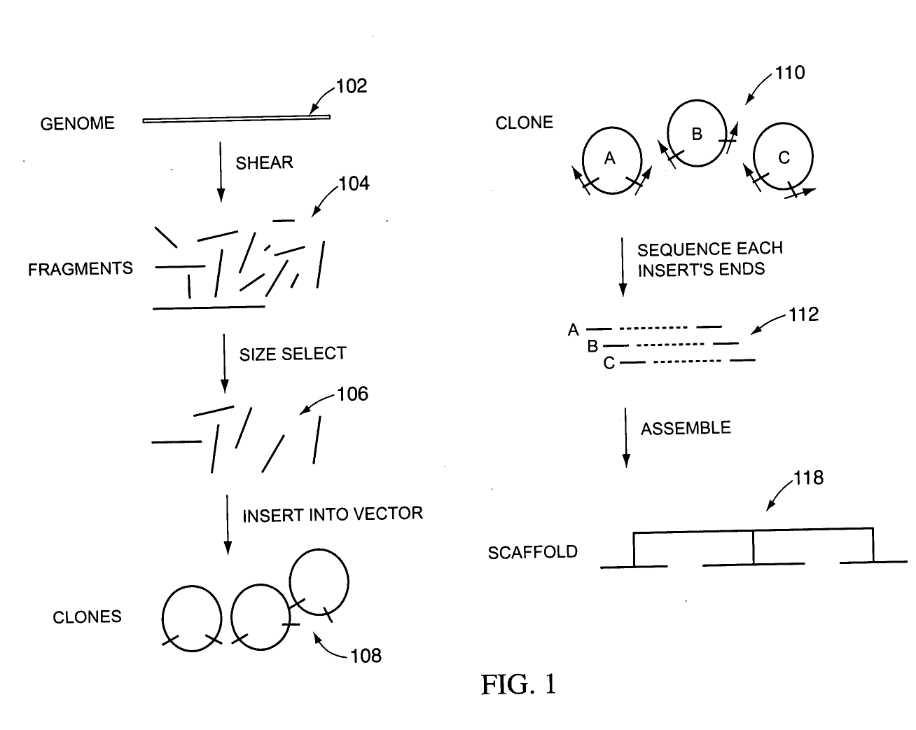

### DESCRIPTION OF VARIOUS EMBODIMENTS DESCRIPTION

The section headings used herein are for organizational purposes only and are not to be construed as limiting the subject matter described in any way.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Figure one illustrates the traditional Shotgun Sequencing technique. First a genetic sequence (102) is sheared to generate fragments (104). These fragments are then size selected to choose fragments that can be conveniently cloned (106). Selected fragments are inserted into vectors and cloned (108). Via sequencing, the first several hundred bases of each end of the insert sequence are determined (110). Next, via sequence assembly techniques, the insert end sequence information and the approximate length of the inserts can be used to determine overlapping fragments (112). These overlapping fragments can then be collapsed into a scaffold. For a more complete description of the process, the reader is referred to U.S. Pat. No. 6,714,874 included by reference in its entirety.

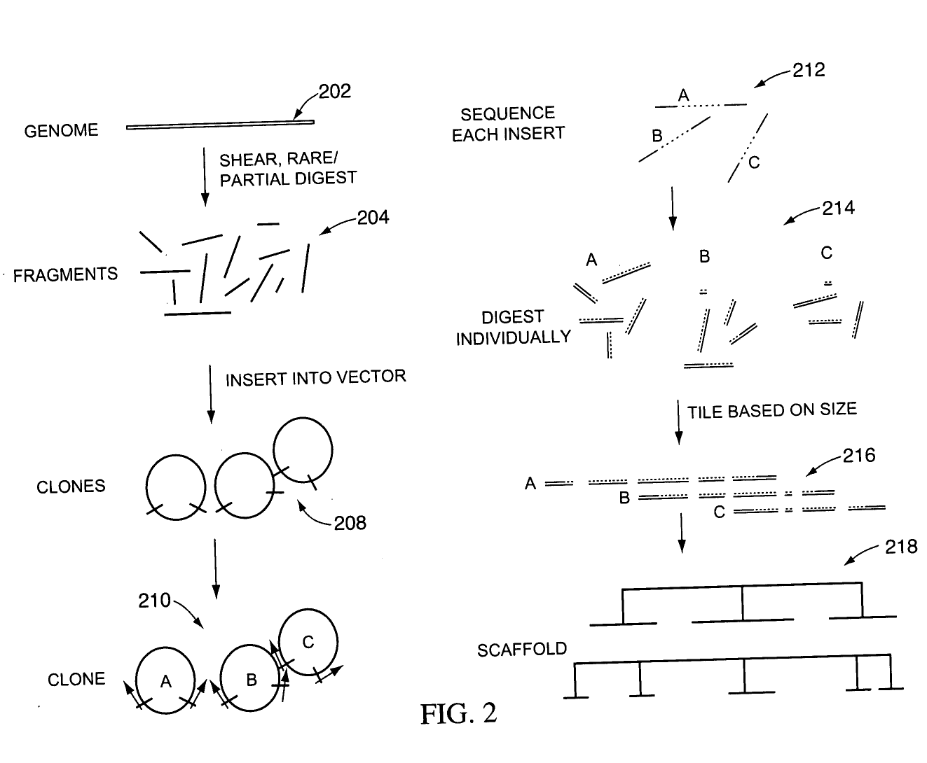

FIG. 2 illustrates an embodiment of the RSSS technique. In 202, genomic DNA is sheared via standard methods, a partial/rare restriction digest or other suitable techniques. This results in a series of fragments (204). These fragments are inserted into vectors (208) and cloned (210). As in the traditional shotgun sequencing method, the ends of the fragments are sequenced (212.) One skilled in the art will appreciate that the present teachings can also be employed with fragments that are fully sequenced. However, it is a fairly typical case that only the ends of fragments can be sequenced reliably. Subsequent to sequencing, the fragments are restriction digested at 214. If two clones (A and B) are adjacent (overlap to some extent), a subset of A's fragment sizes will bear similarity to a subset of B's fragment sizes. This information can be used to generate a restriction fragment map as shown in 216, which can then be collapsed into a scaffold (218.) This process can achieve a “good” assembly using only 2× sequencing (20× clone coverage).

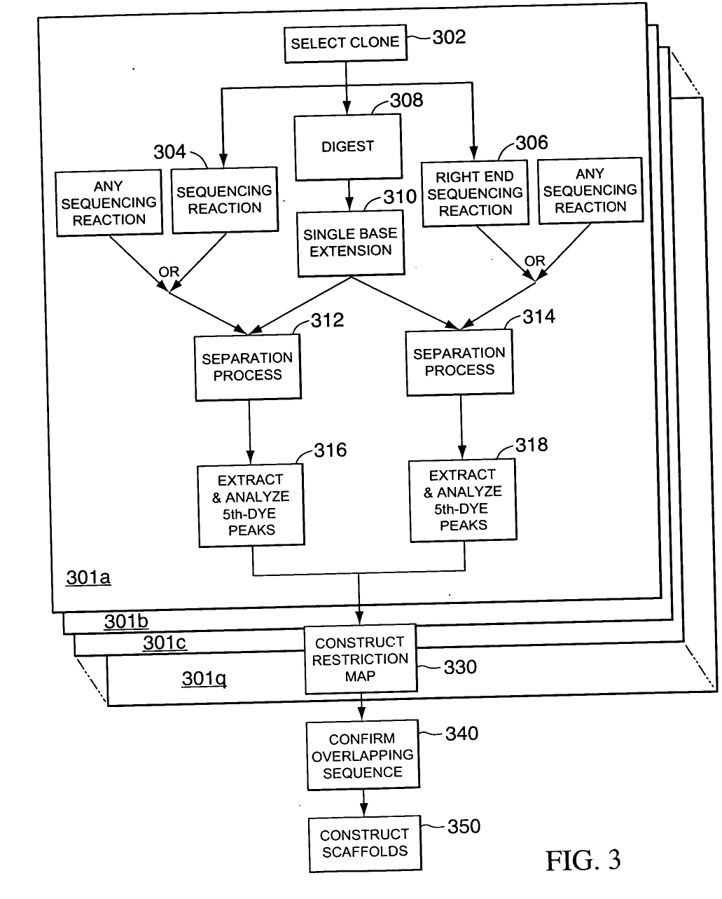

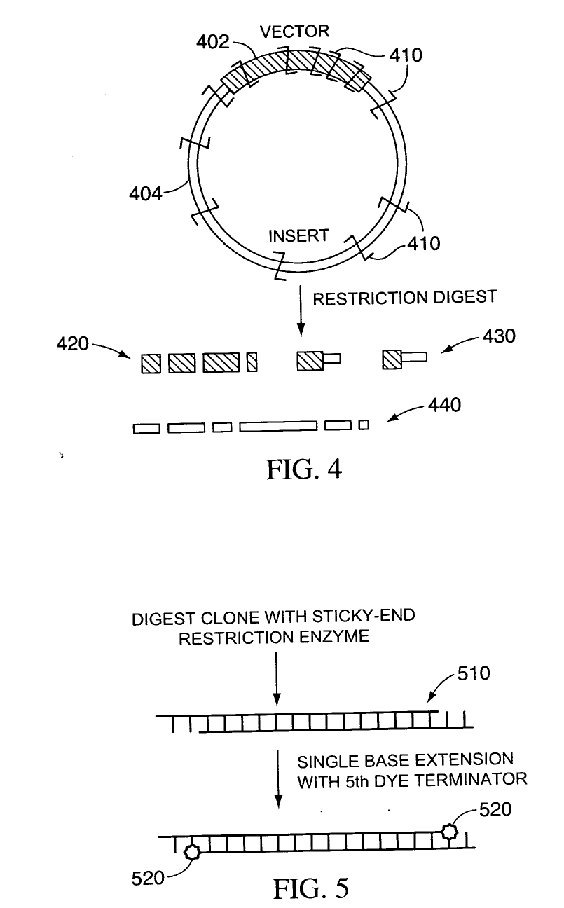

Some embodiments use the process illustrated in FIG. 3. After the clones are generated, the clones of each insert fragment are separated into three portions. Portion one is cycle sequenced from the left-end (304). Portion two is cycle sequenced from the right-end (306). The products of these reactions are kept separate. Portion three is restriction-site digested using a sticky-end cutter (308) which yields three types of fragments. The genesis of these fragments is illustrated in FIG. 4, where 402 identifies the vector sequence, 404 identifies the insert sequence and the multiple 410 elements signify restriction sites. The three types of fragments that results from digestion at the restriction sites are, (1) “type 1” fragments that are wholly contained in the clone vector (420), (2) “type 2” fragments that are wholly contained in a clone insert (440), and (3) “type 3” fragments that span the ends of the clone vector and clone insert (430). At most there can be only two type 3 fragments.

In some embodiments, fragments resulting from the digestion next undergo a labeling process to produce Labeled Digest Fragments (LDF.) Some embodiments use a single-base extension reaction where the ddNTPs used in the reaction have a dye distinguishable from the dye on the other ddNTPs used in the sequencing reaction. The product of the single-base extension reaction is illustrated in FIG. 5. The initial fragment is illustrated at 510. The uneven cutting results in the sticky ends to which the single-base extension ddNTP (520) is incorporated. Some embodiments use the description of a fifth dye as described in U.S. patent application Ser. No. 10/193,776 included by reference herein in its entirety.

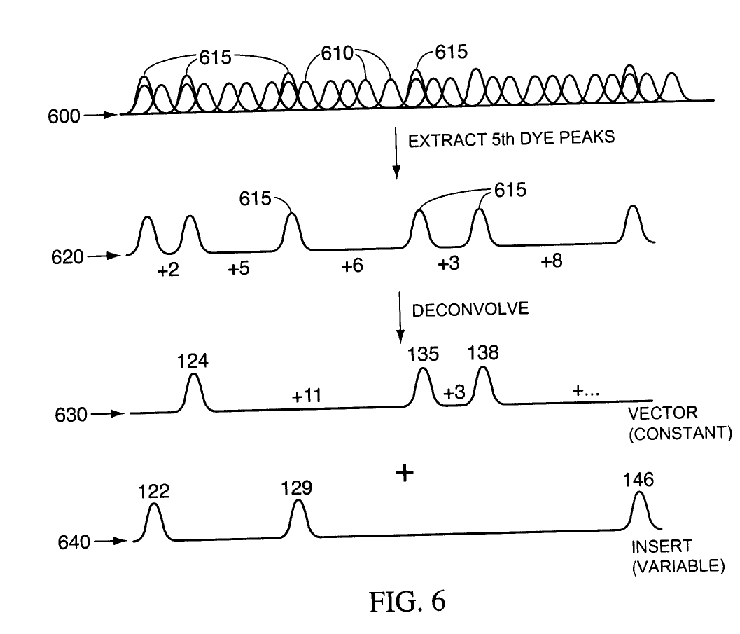

Looking back at FIG. 3, the product of the single-base extension reaction is combined with the product of the left-end sequencing reaction and the fragments are run in a separation medium at 312. One skilled in the art will appreciate that a variety of separation techniques exist. These include gel electrophoresis and capillary electrophoresis. One advantage conferred by running the sequencing product in the same channel as the digestion fragments is the ability to read digestion fragment sizes with single base resolution. The separation yields at least (1) the end read sequence of each end of the clone, and (2) peaks identifying the lengths of the fragments resulting from the restriction digest fragments in the 5th-dye channel. Typically the data appears as in FIG. 6. Here the types of peaks shown at 610 are due to the end sequencing reaction and the types of peaks at 615 are due to the digestion fragments. These later peaks can be extracted from the data using various techniques. One such technique is multicomponenting using the principles as described in U.S. Pat. Nos. 6,015,667 and 6,333,501, both of which are included herein in their entirety. These peaks, once extracted, are shown as a trace in 620. The peaks in 620 are due to the three previously mentioned fragment types. The type 1 fragments (fully contained in the vector) form a distinct signature that is invariant from clone to clone. These peaks are illustrated at 630 and can be subtracted from each LDF dye trace. This leaves the type 2 and type 3 fragments that vary in size from clone to clone but can bear similarity in the case where inserts overlap.

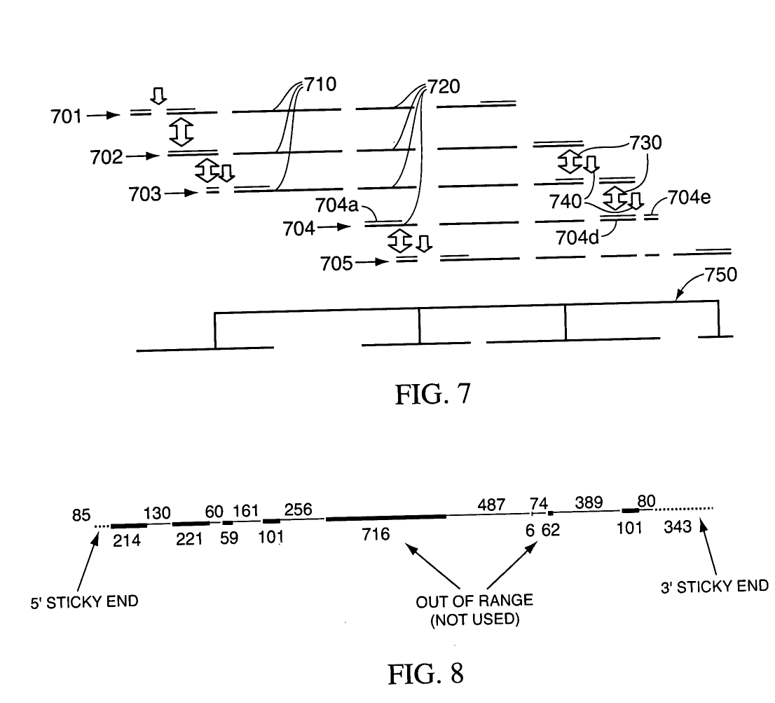

Some embodiments build a restriction map as indicated in 330 and further detailed in FIG. 7. This process is often referred to as “tiling.” Each clone has a list of associated fragment sizes. Some embodiments build a map by determining if there are a significant number of fragments that are of the same size between a set of clones. While all clones and their fragments can be compared to each other clone, this may prove computationally expensive. Some embodiments group clones that are likely to form a contig by first forming a clone family comprised of clones with common fragment sizes. Such grouping assumes that adjacent clones are more likely to share digest fragments, and thus should group together. False grouping from size-coincident digest fragments will increase the noise in a group, however these can introduce non-conforming fragment sizes that will not fit into the group.

Some embodiments group clones for tiling by examining the Total Length of Shared Sizes (TLSS) between clones. The TLSS is the sum of the shared fragment sizes between two clones. Thus two fragments that have a TLSS that exceeds a threshold are designated as overlapping. If a fragment is a candidate for joining a clone family and it does not meet the TLSS threshold, it is rejected. One method of determining a suitable TLSS threshold involves using a complete mammalian genome, and via simulation, determining a value for the TLSS for which there is a high probably that the clones overlap. To accomplish this, some embodiments in silico shear the genome into fragments of the length expected for the cutter that will be used for digestion. For example, a test genome can be generated using a mammalian C4 (created by Celera Genomics for customer use, designated as Release 26) genome sequence with any gaps filled with random scaffold sequences from a pool of mammalian DNA repeats. This results in a 2,861,601,159 base pair sequence. If a cutter that statistically would result in 10 kb fragments will be used, then the sequence can be in silico sheared into clone fragments mean length 10 kb and standard deviation of 1 kb. These inserts can be circularly annealed to a cloning vector such as pBR194c and in silico digested. The pBR194c vector is illustrated in FIG. 8. When digested with a CATG sticky end cutter the vector will produce fragments of length 10, 36, 63, 64, 78, 84, 105, 134, 165, 218, 225,260, 393, 491, and 720. Sizes less than 20 and greater than 550 can be ignored, i.e. 10, and 720 as they can be outside the range of some sequencers. The end fragment sizes are 85 and 343, these will be added to the inserts. The insert fragments will vary in size.

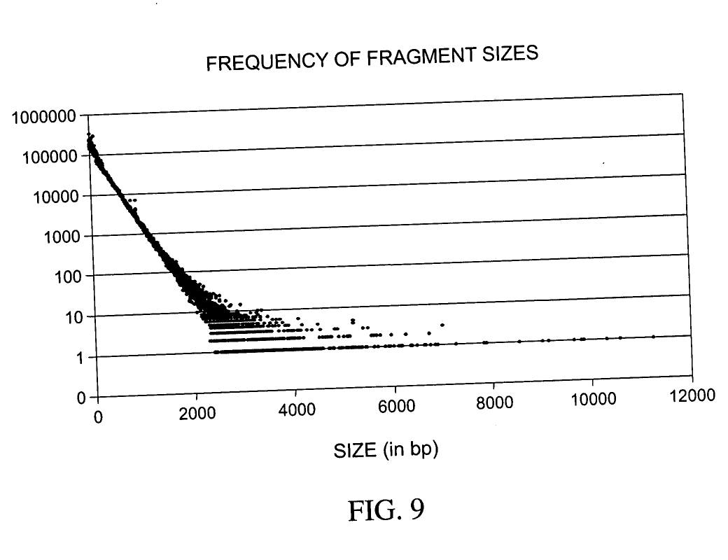

FIG. 9 is a histogram of the resulting insert digest fragments. In total there are approximately 858K insert digest fragment sizes. The largest fragment is 11,083 bp. There are 380K fragments less than 20 bp and 338K fragments greater than 550 bp. Fragments with sizes matching type 1 fragments (wholly contained in the vector sequence) and fragments that are less than 20 bp or greater then 550 bp can be filtered out. This leaves approximately 3,468 K fragments in the resolvable range. Masking out the vector fragments merely simulates subtraction of the peaks corresponding to the type 1 fragments.

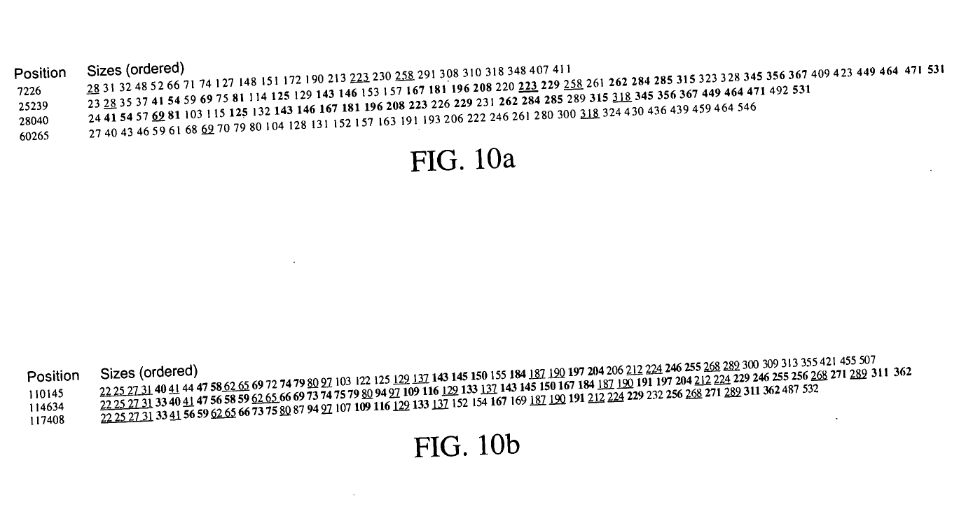

FIG. 10a shows a few of the clone digests from the simulated genome. The clone position is shown in the first column. Between the first pairs and the last pairs of clones, with no effective overlap, very few shared fragments (underlined) are found. The total number of basepairs overlapping are 509 in the first pair and 387 in the second. The inner pair of fragments has 2.8 Kb of overlap and the shared fragments (bolded) are much more frequent. Their sum is 5,638 bp. FIG. 10b shows three clones in close proximity as evidenced by their start positions. Fragments that overlap all three clones are underlined and pair-wise adjacent shared fragments are bolded. Here, the TLSS between all three fragments is 2086 while the all fragments in the middle clone are either shared between all three clones of pair-wise shared by the neighboring clones.

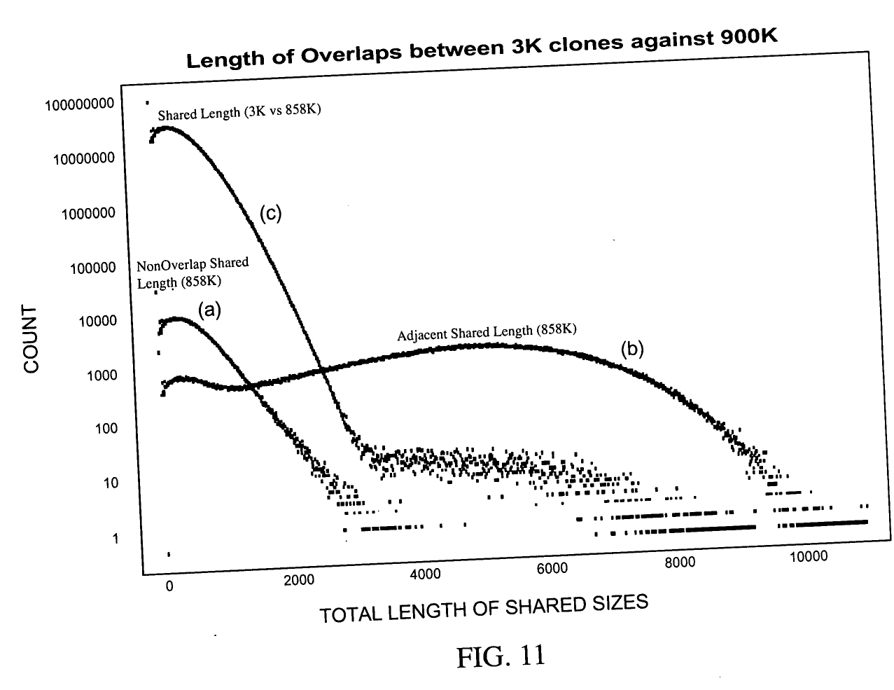

In order to determine a suitable threshold for the total length of shared sizes that can be used to group clones together, some embodiments compute a threshold by graphically determining a threshold beyond which it is not probable that non-overlapping clones would not have an acceptable TLSS. For example, in FIG. 11, trace (a) deals only with known non-overlapping clones taken from the in silico set of approximately 858,000 clones determined above. It plots the number of fragments against the TLSS. After approximately 3 kb total length of shared size fragments, there are virtually no non-overlapping fragments. Thus, for 10 kb clones, one method for determining whether two clones do overlap is to test if they possess more than 3 kb in TLSS. Some embodiments choose a higher, more conservative threshold. Some embodiments may choose a lower number with the realization that more potential misgroupings can occur. One skilled in the art will appreciate that the process can be repeated in order to determine a TLSS threshold for clones of any size. FIG. 11 also contains traces (b) and (c). Trace (b) plots the TLSS for all overlapping clones of the 858k clone set and shows that virtually any amount of TLSS sizes can be expected with some degree of frequency. Trace (c) shows the TLSS for overlap for a randomly selected group of 3,000 clones from the group of 858,000.

Once a set of overlapping clones is identified, the clones can be aligned into a restriction fragment map. FIG. 7 shows five post-digestion clones numbered 701-705 respectively. Sequence information from the sequencing reaction and analysis is indicated by a line on the top of the fragment (710). Same-sized fragments are indicated at 720 and 730. Once the fragments are tiled, sequence information can be used in order to verify the overlap. This is illustrated by the bidirectional arrows at 730. By considering the restriction site information, the type 3 fragments can be correctly placed. For example, in 704a is type two fragment as is 704e. Since the left hand side of 704a does not end at a restriction site, and the sequencing occurs from the outer end towards the center of the DNA, the fragment is oriented as shown. Thus, it can be inferred that fragment 704e belong on the right hand side of the fragment. By looking at the sequence information, it can be inferred which end of the fragment abuts fragment 704d.

One skilled in the art will appreciate that a variety of tiling algorithms exist that can form the basis for the tiling process described herein. For example, the method of Durand (“An efficient program to construct restriction maps from experimental data with realistic error levels”, Nucleic Acids Research v 12:1, 703-716, 1984) can serve as the basis.

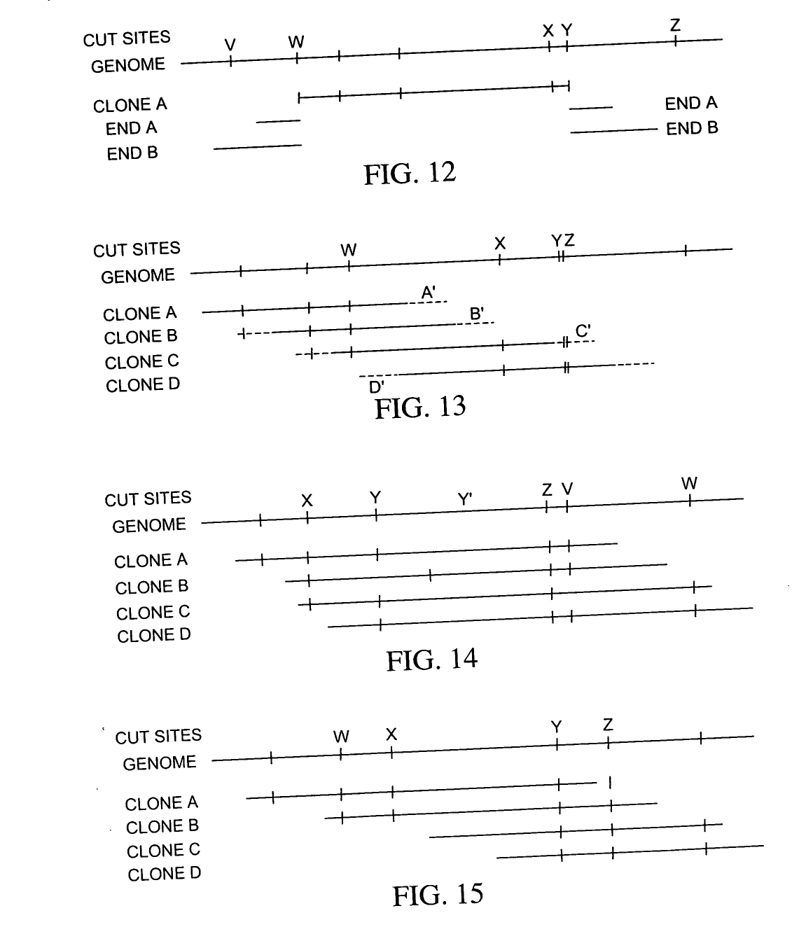

Some embodiments employ logic that considers the length of the insert-end fragments in conjunction with the tiling path to place the insert-end fragments. If a frequent cutter is used in the digestion, it is likely that the sequenced portion of the clone will be cut. This will result in at most two fragments that do not fit the tiling path. If there are more than two fragments, some embodiments can flag the clone as a false join. In FIG. 12, the clone is already part of the tiling path and the two remaining fragments can be oriented either A-clone-B or B-clone-A. The proper orientation can be determined by taking note that fragment B cannot fit between point V and W and thus the proper orientation is A-clone-B. The residual of the restriction site can be used to properly orient the fragment once it is determined on which side of the clone it will be located.

Some embodiments detect the location of fragments greater than the accepted maximum size by taking notice of multiple end fragments that cannot be placed without overlap. For example, in FIG. 13, suppose that the fragment between points W and X is oversized. Now, insert end fragments A′, B′ and D′ cannot be placed without overlapping. If any of the end-sequences (dashed lines) overlap then the gap can be sized. If not, some embodiments place the ends and bound the size of the gap, filling in any unknown sequence with Ns.

Some embodiments recover insert digest fragments (type 1 or type 3) that are masked by the subtracted-out type 2 digest fragments. Short insert digest fragments are likely covered by a sequencing read. Longer fragments can be detected by adding the marked vector fragment sizes one by one to a clone to see if the tiling with its neighbors improves.

Some embodiments account for polymorphisms that either remove a cutting site or add a new one. Logic can be used that recognizes that two-non-conforming fragments can be joined to form a fragment pair that would be the same length as another fragment pair from a clone that does fit the tiling path. These can be combinatorially created from pairs of non-conforming fragments to see if the pairs match a conforming fragment of an adjacent clone. This is illustrated in FIG. 14. Clone B has a polymorphic site at Y′ and hence the fragment YY′ and Y′Z do not fit the tiling path. However, it can be determined that YY′Z is equivalent in length to YZ and hence the two smaller fragments can be placed correctly.

Some embodiments consider the effects of a clone having multiple same-sized fragments. For example, FIG. 15 shows clone B having fragment WX and YZ that are the same size. Thus tiling will remain consistent up to Clone A and from clone C down. Although clone B has passed the TLSS test, it cannot be tiled correctly due to the missing YZ segment which is masked by the WX segment. A check for making the clone tile correctly by using a fragment of the same size can be used alone or can also be used with a check for nearby same-sized fragments on adjacent clones.

Once information in addition to the fragment sizes is used to check the tiling and the orientations of as many fragments as possible, the tiling can be collapsed into a scaffold as indicated in FIG. 7.

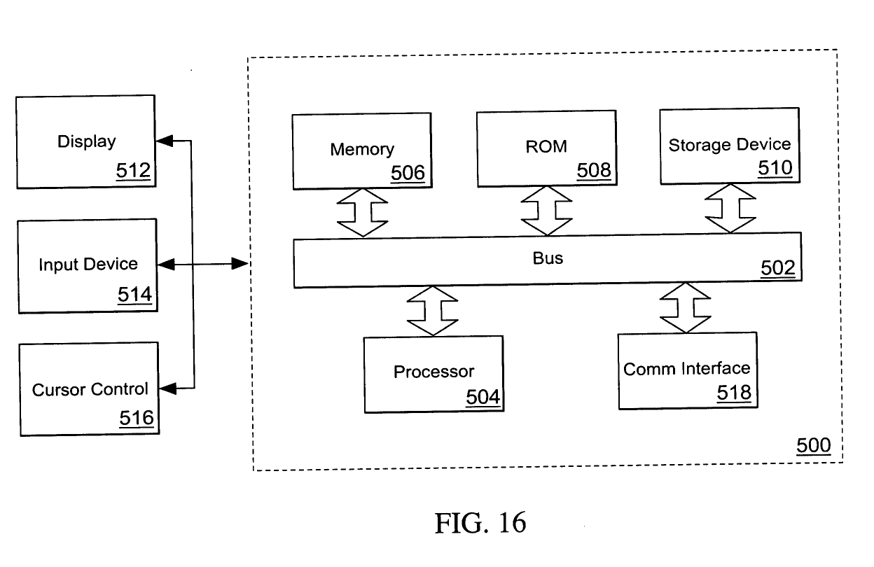

FIG. 16 is a block diagram that illustrates a computer system 500, upon which embodiments of the invention may be implemented. Computer system 500 includes a bus 502 or other communication mechanism for communicating information, and a processor 504 coupled with bus 502 for processing information. Computer system 500 also includes a memory 506, which can be a random access memory (RAM) or other dynamic storage device, coupled to bus 502 for determining base calls, and instructions to be executed by processor 504. Memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT) or liquid crystal display (LCD), for displaying information to a computer user. An input device 514, including alphanumeric and other keys, is coupled to bus 502 for communicating information and command selections to processor 504. Another type of user input device is cursor control 516, such as a mouse, a trackball or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

A computer system 500 can perform the methods described in the present teaching. Consistent with certain implementations of the invention, a consensus sequence or scaffold can be is provided by computer system 500 in response to processor 504 executing one or more sequences of one or more instructions contained in memory 506. Such instructions may be read into memory 506 from another computer-readable medium, such as storage device 510. Execution of the sequences of instructions contained in memory 506 causes processor 504 to perform the process described herein. Alternatively hard-wired circuitry may be used in place of or in combination with software instructions to implement the invention. Thus implementations of the invention are not limited to any specific combination of hardware circuitry and software.

The term “computer-readable medium” as used herein refers to any media that participates in providing instructions to processor 504 for execution. Such a medium may take many forms, including but not limited to, non-volatile media, volatile media, and transmission media. Non-volatile media includes, for example, optical or magnetic disks, such as storage device 510. Volatile media includes dynamic memory, such as memory 506. Transmission media includes coaxial cables, copper wire, and fiber optics, including the wires that comprise bus 502. Transmission media can also take the form of acoustic or light waves, such as those generated during radio-wave and infra-red data communications.

Common forms of computer-readable media include, for example, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 504 for execution. For example, the instructions may initially be carried on the magnetic disk of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector coupled to bus 502 can receive the data carried in the infra-red signal and place the data on bus 502. Bus 502 carries the data to memory 506, from which processor 504 retrieves and executes the instructions. The instructions received by memory 506 may optionally be stored on storage device 510 either before or after execution by processor 504.

The foregoing description of an implementation of the invention has been presented for purposes of illustration and description. It is not exhaustive and does not limit the invention to the precise form disclosed. Modifications and variations are possible in light of the above teachings or may be acquired from practicing of the invention. Additionally, the described implementation includes software but the present invention may be implemented as a combination of hardware and software or in hardware alone. The invention may be implemented with both object-oriented and non-object-oriented programming systems.

All literature and similar materials cited in this application, including but not limited to, patents, patent applications, articles, books, treatises, and internet web pages are expressly incorporated by reference in their entirety for any purpose.

While the present teachings have been described in terms of these exemplary embodiments, the skilled artisan will readily understand that numerous variations and modifications of these exemplary embodiments are possible without undue experimentation. All such variations and modifications are within the scope of the current teachings.

1. A method for determining the sequence of a nucleotide sequence comprising,
generating one or more sets of nucleotide fragments from said nucleotide sequence,
generating one or more sets of end-sequenced fragments by sequencing the ends of the fragments in said one or more sets of nucleotide fragments,
generating one or more sets of restriction-digested fragments by restriction digesting said one or more sets of end-sequenced fragments,
generating one or more tiling sets of fragments from said one or more sets of restriction-digested fragments,
tiling said one or more tiling sets of fragments to form one or more restriction fragment maps,
determining the sequence of said nucleotide sequence by collapsing said one or more restriction fragment maps and aligning sequence information corresponding to the sequenced ends of the fragments in said one or more sets of end-sequenced nucleotides fragments.

2. The method of claim one wherein said first set of nucleotide fragments is generated by random shearing.

3. The method of claim one wherein said first set of nucleotide fragments is generated by partial digestion.

4. The method of claim one further comprising filtering said set of nucleotide fragments in order to select fragments within one or more user-defined size ranges.

5. The method of claim 4 further comprising determining the size of the fragments in said tiling set of fragments.

6. The method of claim 5 wherein said determining comprises
performing a single base extension reaction on said tiling set of fragments,
forming a mixture by combining the product of said single-base extension reaction with the product of a sequencing reaction wherein the ddNTPs in the single-base extension are labeled with different dyes than those used in the single-base extension reaction, and
running said mixture in a separation medium.

7. The method of claim one further comprising,
determining a total length of shared sizes between a first set of restriction-digested fragments and a second set of restriction-digested fragments, and
forming a tiling set of fragments consisting of the fragments from said first and second set of restriction-digested fragments if the total length of shared sequences exceeds a user-defined threshold.

8. A program storage device readable by a machine, embodying a program of instructions executable by the machine to perform method steps for determining the sequence of a nucleotide sequence comprising,
receiving information regarding one or more sets of nucleotide fragments from said nucleotide sequence,
receiving information regarding one or more sets of end-sequenced fragments derived from said one or more sets of nucleotide fragments,
receiving information regarding one or more sets of restriction-digested fragments derived from said one or more sets of end-sequenced nucleotide fragments,
generating one or more tiling sets of fragments from said one or more sets of restriction-digested fragments,
tiling said one or more tiling sets of fragments to form one or more restriction fragment maps,
determining the sequence of said nucleotide sequence by collapsing said one or more restriction fragment maps and aligning sequence information corresponding to the sequenced ends of the fragments in said one or more sets of end-sequenced nucleotides fragments.

9. The program storage of claim eight further comprising filtering said set of nucleotide fragments in order to select fragments within one or more user-defined size ranges.

10. The method of claim eight further comprising,
determining a total length of shared sizes between a first set of restriction-digested fragments and a second set of restriction-digested fragments, and
forming a tiling set of fragments consisting of the fragments from said first and second set of restriction-digested fragments if the total length of shared sequences exceeds a user-defined threshold.
